(19) United States
(12) Patent Application Publication
(10) Pub. No.: US 2026/0363764 A1
(43) Pub. Date: Nov. 19, 2026

(54) FLEXIBLE INTERNALLY COOLED RADIOFREQUENCY PROBE

(71) Applicant: Baylis Medical Technologies Inc., Mississauga (CA)

(72) Inventor: Neil Godara, Milton (CA)

(21) Appl. No.: 19/677,318

(22) Filed: May 14, 2026

Related U.S. Application Data
(60) Provisional application No. 63/806,797, filed on May 16, 2025.

(51) Int. Cl. A61B 18/14; A61B 18/00

(52) U.S. Cl. CPC A61B 18/1492; A61B 2018/00023; A61B 2018/00577; A61B 2018/00821

(57) ABSTRACT

A flexible cooled probe for use ablation of spinal nerves, tumor ablation in the spine or periphery, or interventional pain management procedures. The probe having a shaft assembly comprising two internally reinforced coolant tubes, a distal tip with bipolar electrodes, sealed to the tubing, a hypotube with embedded thermocouple wires for temperature monitoring, a surrounding shaft insulation layer for electrical and thermal containment, a handle assembly for fluid and electrical connections. The probe forming part of a larger system comprising a peristaltic pump for closed-loop coolant circulation and a radiofrequency generator capable of bipolar energy delivery.

### TECHNICAL FIELD

The disclosure relates to medical devices, and more particularly to a cooled probe for ablating a target tissue using radiofrequency energy.

Existing cooled radiofrequency (“RF”) ablation systems often rely on rigid shafts, external cooling sleeves, or multiple assembly layers that make them difficult to use in anatomically constrained or curved access paths. These systems can suffer from limited flexibility, poor thermal control, and complex construction. Additionally, many bipolar RF probes lack internal cooling, reducing their efficacy and increasing the risk of thermal injury to surrounding tissues.

### BRIEF DESCRIPTION OF THE DRAWINGS

In order that the invention may be readily understood, embodiments of the invention are illustrated by way of examples in the accompanying drawings, in which:

FIG. 1 is an illustration of a system comprising a cooled radiofrequency probe in accordance with an embodiment of the present invention; and

FIGS. 2A and 2B are cross sections taken through a distal portion of the elongate member of the probe of FIG. 1, in accordance with an embodiment of the present invention.

In the drawings, like reference characters generally refer to the same parts throughout the different views. Also, the drawings are not necessarily to scale, emphasis instead being placed upon generally illustrating the various concepts discussed herein.

### DETAILED DESCRIPTION

The inventors have developed a novel design for cooled bipolar radiofrequency (“RF”) probe construction, with improved flexibility, a reduced number of assembly steps, and robust high-pressure cooling performance. Existing designs often use rigid assemblies or rely on cooling jackets or external sleeves, which are bulky and difficult to scale down for applications requiring fine, curved access.

In one broad aspect of the present invention, a probe for delivering energy to a target tissue is provided, the probe comprising: an elongate member, the elongate member defining a lumen configured for receipt of a cooling fluid; at least one electrode for delivering ablative energy to a target tissue, the at least one electrode associated with a distal region of the elongate member; at least one temperature sensor associated with the elongate member for measuring a temperature within the target tissue; and a plurality of flexible tubes located at least partially within the lumen for delivering the cooling fluid.

In some embodiments, each of the flexible tubes comprises a reinforcing structure which may be, for example, a hypotube or a metallic braid.

In addition, in some embodiments, the tubes are fabricated from a plastic or polymeric material to provide flexibility. The tubes may comprise both an inflow and an outflow tube.

In some embodiments of the present invention, the integration of flexible coolant delivery tubing directly into the functional distal electrode assembly, eliminates the need for separate structural supports or external flow channels. The tubing may connect, for example, directly into a metallic segment brazed onto a distal electrode, forming both a mechanical and hydraulic connection. The present invention simplifies the interface between flexible components and rigid conductive elements, enabling a lower-profile, more manufacturable design with improved fluid control and electrical reliability.

Embodiments of the present invention deliver RF energy through a flexible bipolar probe while actively cooling the one or more electrodes via an internal flow path for circulation of a cooling fluid. For example, a peristaltic pump delivers fluid to the probe through reinforced, flexible tubing. This tubing connects directly (for example via brazing) to a metal distal section that functions as an electrode, creating a secure fluid channel within the ablation tip. The return coolant flows back through a second internal reinforced tube.

This integrated design allows: i) cooling of both electrodes during energy delivery; ii) flexibility along the shaft for, for example, curved anatomical access; iii) improved manufacturability and reliability due to the brazed tubing-to-electrode interface; and iv) embedded temperature sensing via a thermocouple in a hypotube welded to the distal tip, enabling closed-loop energy control.

The combination of cooling, sensing, and RF delivery in a single, compact shaft addresses the limitations of current rigid or multi-component systems.

The flexible probe/system may be used in at least the following applications: ablation of spinal nerves (e.g., basivertebral nerve, medial branch nerves), tumor ablation in the spine or periphery, interventional pain management procedures, office-based or ambulatory RF treatments requiring flexible catheter delivery, or any other applications where existing cooled RF systems are too rigid or bulky.

In one embodiment, the flexible shaft assembly comprises: two internally reinforced coolant tubes (inflow and return); a distal tip with bipolar electrodes, sealed to the tubing; a hypotube with embedded thermocouple wires for temperature monitoring; a surrounding shaft insulation layer for electrical and thermal containment; a handle assembly for fluid and electrical connections; a peristaltic pump for closed-loop coolant circulation; and an RF generator capable of bipolar energy delivery.

These components are integrated to allow for thermal regulation, consistent electrode performance, and user-friendly mechanical design.

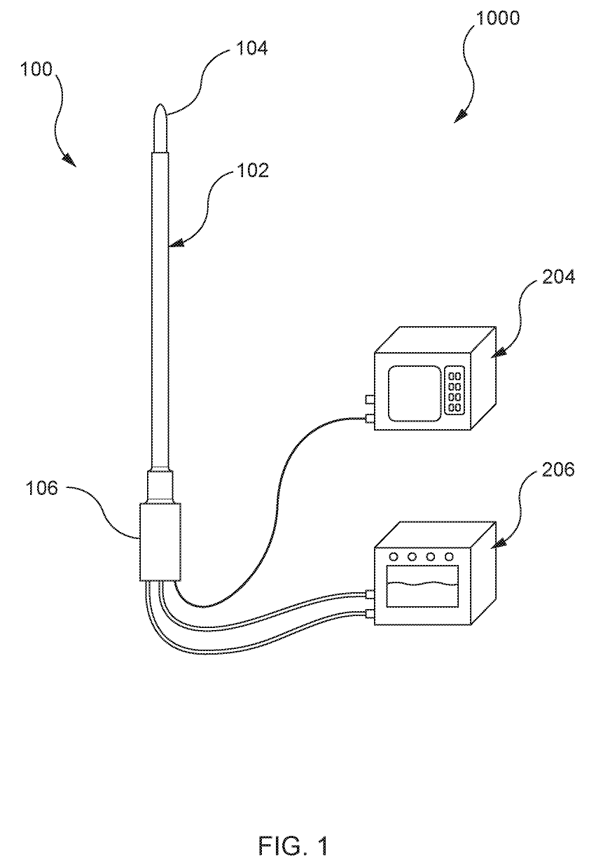

Referring to FIG. 1, according to one embodiment of the present invention, the ablation system 1000 comprises a probe 100 having an elongate member 102 and active electrode 104 for delivering ablative energy. The probe is connected to a radiofrequency generator 204 and a fluid circulation system 206. In some embodiments, the system also includes a grounding pad (not shown). In other embodiments, the probe 100 is a bipolar probe 100. Probe 100 comprises a handle 106 and has various wires, cables, and tubes for delivering energy and fluid to the probe 100.

In one embodiment, fluid circulation system 206 includes a peristaltic pump that drives water through the inlet tube and draws it back via the outlet tube. Flow rate and pressure are adjustable. The system is tested to withstand continuous operation at 100 psi with proper cooling.

Radiofrequency generator 204 provides bipolar energy delivery across the electrodes. The design is compatible with standard clinical RF generators, but optimized for systems with cooling feedback control.

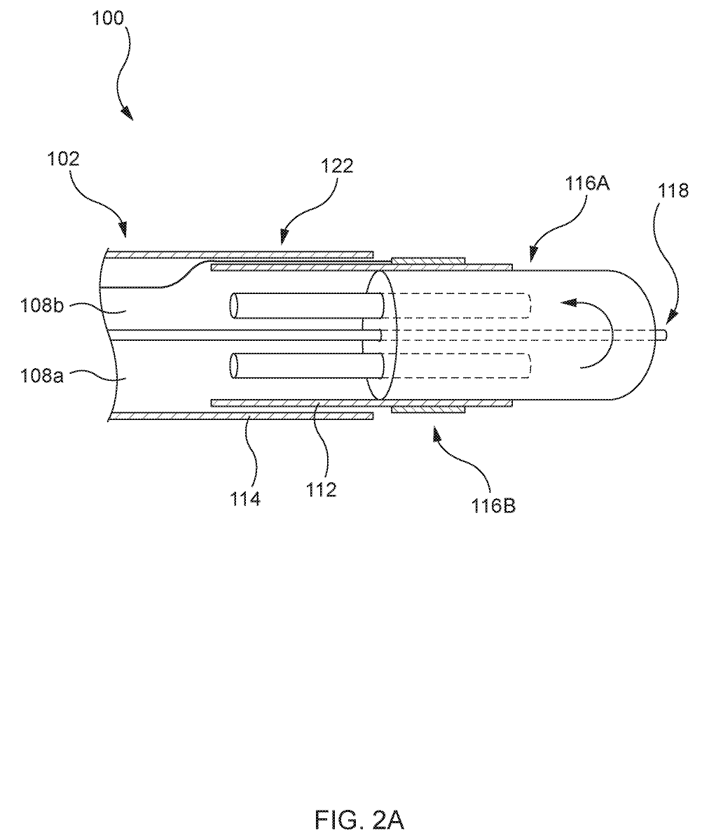

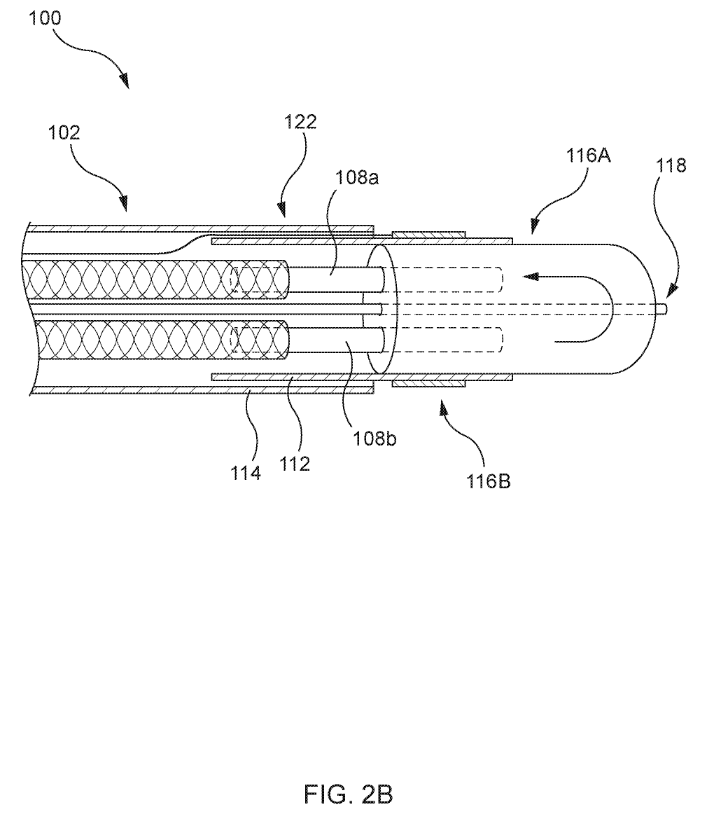

Referring to FIGS. 2A and 2B, in one embodiment, probe 100 comprises an elongate member 102 (also referred to as “shaft 102”). Shaft 102 comprises a flexible plastic extrusion 122, capable of housing two cooling tubes 108a, 108b and a reinforcing structure 110 (for example, a hypotube or a braid). To withstand internal pressure generated by the coolant system (which may be, for example, up to 100 psi), the shaft is over-jacketed with a braided polymer sleeve 112 (e.g., PET, aramid, or nylon braid), then covered with a thermoplastic outer jacket 114 (e.g., Pebax, polyurethane, or nylon) for flexibility and durability. In some instances, the outer shaft need not withstand the internal pressure as the cooling tubes 108a, 108b are sufficiently reinforced.

The outer jacket 114 maintains axial and radial strength without compromising flexibility and ensures kink resistance and containment under pressure.

Two internal flexible plastic tubes 108, one for coolant inflow (108a) and one for outflow (108b), are arranged in parallel and run along the shaft 102. In one embodiment, each is constructed from reinforced medical-grade polymer tubing (e.g., Pebax, polyimide, or nylon) with embedded braided reinforcement 110 (such as stainless steel or polyester fibers) for burst resistance. An inner diameter of each tube 108 is sufficient for flow rates of, for example, between about 10 to about 30 mL/min. Each tube 108 may be capable of handling 100 psi of pressure.

The electrode tip 104 of the distal end of the probe 102 features bipolar electrodes 116a and 116b, which may be made from stainless steel, platinum-iridium, or nickel-titanium. In some embodiments, electrode 116a is a distal electrode and electrode 116b is a proximal electrode. Distal electrode 116a may be brazed, laser-welded or soldered to the ends of the coolant tubes. Further, it may be fluid-sealed to prevent coolant leakage. Proximal electrode 116b may be, for example, glued or swaged onto sleeve 112 or shaft 102. Electrodes 116a and 116b may be configured for bipolar energy delivery and may be connected to handle 106 via one or more wires that extend along the shaft or within a lumen of the shaft.

In one embodiment, probe 100 comprises a temperature sensor 118, for example a thermocouple 108. In one embodiment, thermocouple 108 is a Type K or J thermocouple that runs through a small-diameter stainless steel hypotube 120 (e.g., 25–27 gauge), which provides structural reinforcement and welding compatibility with the distal tip 104. This tube also assists in maintaining longitudinal stiffness.

In one embodiment, handle 106 is located at the proximal end of the probe 100 and houses connections for inflow/outflow tubing 108, with secure barbed or luer interfaces. It may also comprise solder joints for thermocouple and RF conductors, and a strain-relieved cable exit for connection to the RF generator 206.

In alternate embodiments, the reinforced tubing could be braided tubing, or co-extruded lines. In some embodiments, the thermocouple comprises directly embedded wires without a hypotube. In some embodiments, the probe is a monopolar probe and is grounded externally, for example using a separate grounding pad. In some embodiments, the peristaltic pump may be replaced by a syringe pump or a gravity fed loop. In some embodiments, the shaft jacket may be braided or comprise a molded polymer overwrap. The cooling tubes may be comprised of Nitinol, polymide, pebax, PTFE, or stainless steel.

The embodiment(s) of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention.

1. A probe for delivering energy to a target tissue, the probe comprising:
an elongate member, the elongate member defining a lumen configured for receipt of a cooling fluid;
at least one electrode for delivering ablative energy to a target tissue, the at least one electrode associated with a distal region of the elongate member;
at least one temperature sensor associated with the elongate member for measuring a temperature within the target tissue; and
a plurality of flexible tubes located at least partially within the lumen for delivering the cooling fluid.

2. The probe of claim 1, wherein each of the flexible tubes comprises a reinforcing structure.

3. The probe of claim 2, wherein the reinforcing structure comprises a hypotube.

4. The probe of claim 2, wherein the reinforcing structure comprises a metallic braid.

5. The probe of claim 1, wherein the tubes are fabricated from a plastic or polymeric material.

6. The probe of claim 1, wherein the plurality of flexible tubes comprise an inflow tube and an outflow tube.

7. The probe of claim 1, wherein the plurality of flexible tubes are mechanically coupled to one or more of the at least one electrodes.

8. The probe of claim 7, wherein the plurality of flexible tubes are brazed to a distal electrode of the at least one electrodes.
